(19) United States
(12) Patent Application Publication
(10) Pub. No.: US 2008/0146843 A1
(43) Pub. Date: Jun. 19, 2008

(54) Methods for reducing 7/9-nitrotetracycline derivatives

(75) Inventors: Evgeny Tsiperman, Beer-Sheva (IL); Sergei Fine, Qiriat-Arbaa (IL); Sofia Gorohovsky, Beer Sheva (IL); Slavik Yurkovski, Kiryat Gat-Malkei (IL)

Correspondence Address: KENYON & KENYON LLP, ONE BROADWAY, NEW YORK, NY, 10004, US

(73) Assignee: Teva Pharmaceutical Industries Ltd., Petach Tikva (IL)

(21) Appl. No.: 11/803,142

(22) Filed: May 10, 2007

Related U.S. Application Data
(60) Provisional application No. 60/799,550, filed on May 10, 2006.

(51) Int. Cl. C07C 231/10

(52) U.S. Cl. 564/157; 564/167

(57) ABSTRACT

The invention is directed to processes for the reduction of tetracycline intermediates having a NO2 group.

### CROSS-REFERENCE TO RELATED APPLICATIONS

This application claims the benefit of the filing date of U.S. Provisional Patent Application No. 60/799,550 filed May 10, 2006, the disclosure of which is hereby incorporated herein by reference.

### FIELD OF THE INVENTION

The invention is directed to processes for the reduction of tetracycline intermediates having a NO2 group. Specifically, 7- or 9-nitrotetracycline is reduced to the corresponding 7- or 9-aminotetracycline derivative.

### BACKGROUND OF THE INVENTION

Tigecycline (CAS 220620-09-7), (4S,4aS,5aR,12aS)-9-(2-(tert-butylamino)acetamido)-4,7-bis(dimethylamino)-1,4,4a,5,5a,6,11,12a-octahydro-3,10,12,12a-tetrahydroxy-1,11-dioxo-2-naphthacenecarboxamide, is the first drug of a new generation of tetracycline antibiotics called glycylcyclines. Tigecycline has a wider range of bioactivity than the parent tetracycline and its analogues discovered so far, and it may be administrated less frequently and/or in lower doses.

Tigecycline has been introduced and marketed by Wyeth under the brandname TYGACIL® and it is especially indicated against acute lethal infections caused by organisms carrying tetracycline resistance determinants. TYGACIL® is marketed as a leophilized powder or cake for intravenous injection and the drug substance does not contain excipients or preservatives.

Tigecycline has the following structure:

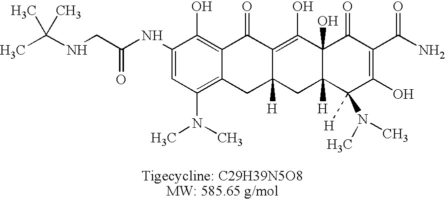

Tigecycline is disclosed in U.S. Pat. No. 5,494,903. Preparation of glycylcyclines requires the use of 9-aminotetracyclines, which are key intermediates for creating this type of antibiotics [Sum, P. E., Petersen, P. Bioorg. Med. Chem. Lett., (1999) 9(10), 1459]. Often, 9-aminotetracyclines are obtained by means of chemoselective reduction of the corresponding nitro compound (J. Am. Chem. Soc., 1960, 82, 1253; J. Med. Chem., 1962, 5(3), 538; J. Med. Chem., 1994, 37, 184; EP 0 535 346, U.S. Pat. No. 5,248,797, U.S. Pat. No. 5,401,863). Chemoselective reduction of the nitro group to the corresponding amino group is a well studied methodology and various reagents and methods are available for this transformation, for example, as disclosed in March's Advanced Organic Chemistry: Reactions, Mechanisms, and Structure; 5th Ed. and references therein.

During preparation of Tigecyline, the 9-nitrotetracycline, (4S,4aS,5aR,12aS)-9-nitro-4,7-bis(dimethylamino)-1,4,4a,5,5a,6,11,12a-octahydro-3,10,12,12a-tetrahydroxy-1,11-dioxo-2-naphthacenecarboxamide, having the following structure:

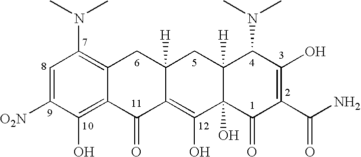

is converted into the corresponding 9-aminotetracycline, (4S,4aS,5aR,12aS)-9-amino-4,7-bis(dimethylamino)-1,4,4a,5,5a,6,11,12a-octahydro-3,10,12,12a-tetrahydroxy-1,11-dioxo-2-naphthacenecarboxamide, having the following structure:

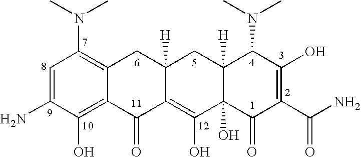

Tetracyclines, in general, and Tigecycline specifically, are very sensitive to various factors including acidity, exposure to light, and exposure to heat which may cause relatively rapid degradation that result in formation of numerous impurities like oxidation and hydrolysis products, such as the C4-epimer.

Most of the processes of the prior art result in a significant amount of the 4-epimer impurity and require the use of an ether, a solvent not suitable for industrial production. In an improved process using catalytic hydrogenation in acidic methanol, ethanol, glycol ethers, or mixtures thereof, the product contains a reduced amount of the 4-epimer impurity. However, glycol ethers are is highly toxic and therefore unsuitable for application on an industrial level.

Thus, there is a need in the art for industrially applicable means for reducing the NO2 group of tetracycline intermediates, specifically 7- or 9-nitrotetracycline, to the corresponding 7- or 9-aminotetracycline. The process of this invention provides such a process.

### SUMMARY OF THE INVENTION

In one embodiment, the present invention encompasses a process for reducing 7- or 9-nitrotetracycline into its corresponding 7- or 9-aminotetracycline comprising: a) providing a mixture of nitrotetracycline in a polar solvent selected from the group consisting of water, C₂₋₆ linear or branched-chain aliphatic alcohols, which may be substituted or unsubstituted (examples of substituted alcohols include diols and their monoethers, and polyglycols); and b) admixing formic acid or a salt thereof and a catalyst with this mixture to obtain 7- or 9-aminotetracycline.

In another embodiment, the obtained 7- or 9-aminotetracycline is recovered.

Preferably 9-aminotetracycline can subsequently be converted to Tigecycline.

Preferably, the Tigecycline obtained by the process of the present invention contains less than about 10%, more preferably less than about 8%, even more preferably less than 6%, and most preferably less than about 3% of the 4-epimer.

### DETAILED DESCRIPTION

In one embodiment, the present invention encompasses a process for reducing 7- or 9-nitrotetracycline into the corresponding 7- or 9-aminotetracycline comprising: a) providing a mixture of nitrotetracycline in a polar solvent selected from the group consisting of water, C₂₋₆ linear or branched-chain aliphatic alcohols, which may be substituted or unsubstituted, (examples of substituted alcohols include diols and their monoethers and polyglycols); and b) admixing formic acid or a salt thereof and a catalyst with this mixture to obtain 7- or 9-aminotetracycline.

In another embodiment, the obtained 7- or 9-aminotetracycline is recovered. Recovery of the aminotetracycine of this invention may be by any means known to the skilled artisan including precipitation, extraction, and chromatography.

In a preferred embodiment, the nitrotetracycline is a 7- or 9-nitrosancycline and the corresponding aminotetracycline is 7- or 9-aminosancycline

In a more preferred embodiment, the nitrotetracycline is a 9-nitrominocycline and the corresponding aminotetracycline is 9-aminominocycline

The 7- or 9-nitrotetracycline mixture may be a suspension or solution, preferably a solution. The alcohols may include diols, mono- or di-ethylene glycols, and monoalcohol ethers.

The polar solvent is preferably water or methanol. Preferably, the volume/weight ratio of the polar solvent to the 7- or 9-nitrotetracycline is about 2 to about 20, preferably the volume/weight ratio is about 3 to about 10.

Suitable catalysts include Raney Nickel and noble metal catalysts, such as platinum and palladium. Preferably, the noble metal catalyst is palladium.

The noble metal catalyst may be provided on an inert support such as carbon, activated carbon, alumina, or an inert inorganic salt. Preferably, the noble metal catalyst is palladium on carbon (“Pd/C”). Preferably, the noble metal catalyst is an amount of about 0.2% to about 20% relative to the amount of 7- or 9-nitrotetracycline, more preferably in an amount of about 1% to about 10%, yet more preferably in an amount of about 2% to about 5%, and most preferably in an amount of about 5% of the active substance. Most preferably, the palladium on carbon is in an amount of about 5% relative to said amount of 7- or 9-nitotetracycline. The catalyst may be removed by any suitable method, including filtration.

Preferably, the reduction is performed in an inert atmosphere, such as nitrogen, to prevent possible oxidation and thus, improve the quality of the product.

In addition, there is a correlation between the initial pH and the impurity profile of the product and thus, one skilled in the art can accordingly increase or decrease the pH, depending on the substrate used, for optimal reaction conditions. For example, when 9-nitrominocycline is used as a substrate, reduction is preferably carried out under about neutral conditions.

The term “formic acid” means formic acid and the salts thereof. The formic acid is preferably ammonium formate, sodium formate, and potassium formate, more preferably the formic acid is ammonium formate. The formic acid is preferably added prior to the addition of the catalyst, although adding the catalyst first is also acceptable.

Preferably, the reduction is carried out to completion while being monitored by periodic TLC or HPLC analysis to determine the end of the reaction, i.e. the disappearance of the starting material.

Preferably, 9-aminotetracycline can subsequently be converted to Tigecycline by any means known in the art, such as disclosed in U.S. Pat. No. 5,675,030.

Preferably, the resulting Tigecycine contains less than about 10%, more preferably less than about 8%, even more preferably less than about 6%, and most preferably less than about 3% of the 4-epimer.

Having thus described the invention with reference to particular preferred embodiments and illustrative examples, those in the art would appreciate modifications to the invention as describes and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinal skill in the art and are described in numerous publications. All references mentioned herein are incorporated in their entirety.

### EXAMPLES

Instrumentation

### HPLC Method

Column stationary phase and dimensions: Discovery RP Amide C16 250×4.6 mm 5μ

Mobile phase: Gradient of Eluent A and Eluent B

| | |
|---|---|
| Eluent “A”: | 0.1% TFA_{(aq)} pH 2.5 by NH₄OH |
| Eluent “B”: | 0.1% TFA v/v in Acetonitrile |

| | | |
|---|---|---|
| Time | % “A” | % “B” |
| 0 | 95 | 5 |
| 15 | 90 | 10 |
| 35 | 30 | 70 |

| | |
|---|---|
| Wavelength: | 244 nm |
| Flow: | 1 mL/min |
| Run time: | 35 min |
| Sample preparation: | 1 mg/mL of sample dissolved in water. |

### Example 1

5 g of crude 9-nitrominocycline (prepared according to J. Med. Chem., 1994, 37, 184) was dissolved in 20 ml of water at ambient temperature. 2 grams of Pd/C 5% were introduced to the dark-brown solution. 1.25 g of ammonium formate was then added and the mixture stirred for 1.5 h (disappearance of the starting material during this time was monitored by HPLC method). The reaction mixture was filtered and the cake washed with 10 ml of water. A combined filtrate was introduced dropwise into 250 ml of isopropanol and the resulting suspension was stirred overnight at ambient temperature. The filtrate was washed with 20 ml of isopropanol and, finally, dried in a vacuum oven at 40° C. overnight, affording crude 9-aminominocycline (chromatographic purity of 73% area, 7.2% area 4-epimer).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A process for reducing 7- or 9-nitrotetracycline into its corresponding 7- or 9-aminotetracycline comprising: a) providing a mixture of nitrotetracycline in a polar solvent selected from the group consisting of water, C2-6 linear or branched-chain aliphatic alcohols and mixtures thereof; and b) admixing formic acid and a catalyst with said mixture.

2. The process of claim 1, wherein said alcohol is selected from the group consisting of diols, substituted alcohols, monoethers, monoglycols, and diglycols.

3. The process of claim 1, further comprising the step of recovering said 7- or 9-aminotetracycline.

4. The process of claim 3, wherein said recovery is selected from the method consisting of precipitation, extraction, and chromatography.

5. The process of claim 1, wherein said nitrotetracycline is a 7- or 9-nitrosancycline and said corresponding aminotetracycline is 7- or 9-aminosancycline.

6. The process of claim 1, wherein said nitrotetracycline is a 9-nitrominocycline and the corresponding aminotetracycline is 9-aminominocycline.

7. The process of claim 1, wherein said mixture is in the form of a suspension or solution.

8. The process of claim 7, wherein said mixture is in the form of a solution.

9. The process of claim 1, wherein said polar solvent is selected from the group consisting of water and methanol.

10. The process of claim 1, wherein a volume/weight ratio of said polar solvent to said 7- or 9-nitrotetracycline is about 2 to about 20.

11. The process of claim 10, wherein said volume/weight ratio of said polar solvent to said 7- or 9-nitrotetracycline is about 3 to about 10.

12. The process of claim 1, wherein said catalyst is selected from the group consisting of Raney Nickel and noble metal catalysts.

13. The process of claim 12, wherein said noble metal catalyst is palladium.

14. The process of claim 12, wherein said noble metal catalyst is platinum.

15. The process of claim 12, wherein said noble metal catalyst is provided on an inert support.

16. The process of claim 15, wherein said inert support is selected from the group consisting of carbon, activated carbon, aluminum, and an inert organic salt.

17. The process of claim 16, wherein said noble metal catalyst is palladium on carbon.

18. The process of claim 12, wherein said noble metal catalyst is present in an amount of about 0.2% to about 20% relative to an amount of 7- or 9-nitrotetracycline.

19. The process of claim 18, wherein said noble metal catalyst is present in an amount of about 1% to about 10% relative to said amount of 7- or 9-nitrotetracycline.

20. The process of claim 19, wherein said noble metal catalyst is present in an amount of about 2% to about 5% relative to said amount of 7- or 9-nitrotetracycline.

21. The process of claim 17, wherein an amount of said palladium on carbon is about 5% relative to an amount of 7-or 9-nitrotetracycline.

22. The process of claim 1, wherein said process is performed in an inert atmosphere.

23. The process of claim 22, wherein said inert atmosphere is nitrogen.

24. The process of claim 1, wherein said formic acid is added prior to said addition of said catalyst.

25. The process of claim 1, wherein said formic acid is selected from the group consisting of ammonium formate, sodium formate, and potassium formate.

26. The process of claim 25, wherein said formic acid is ammonium formate.

27. The process of claim 1, further comprising conversion of said 9-aminotetracycline to Tigecycline.

28. The process of claim 27, wherein said converted Tigecycline contains less than about 10% of a 4-epimer.

29. The process of claim 28, wherein said converted Tigecycline contains less than about 8% of said 4-epimer.
